(19) United States
(12) Patent Application Publication
(10) Pub. No.: US 2020/0305515 A1
(43) Pub. Date: Oct. 1, 2020

(54) Attachable panties that promotes safety and attachable absorbency panels

(71) Applicant: Senwee Bradley, Thousand Oaks, CA (US)

(72) Inventor: Senwee Bradley, Thousand Oaks, CA (US)

(21) Appl. No.: 16/364,205

(22) Filed: Mar. 26, 2019

(51) Int. Cl. A41B 9/00; A61F 13/49

(52) U.S. Cl. CPC A41B 9/001; A61F 13/49004; A61F 13/49017; A61F 13/49011; A61F 13/49006

(57) ABSTRACT

A sanitary underwear with a detachably attachable organic pad is illustrated. The underwear comprises a side fastener having one piece adjustable organic pad extending from the front center belly button and passing through the center of the middle crotch area up to the back center to the waistband. The underwear with the organic pad is enabled to absorb leaks, perspiration, menstrual fluids, urinary, and rectum discharges. The underwear promotes fair wages and trade. The organic pad is made of organic cotton, hemp, bamboo, organic terry cloth, jute and any combination thereof. The underwear provides a plurality of benefits including a reduced risk of carcinogens caused by harmful pesticides and chemicals, promotion of sustainable environment, eco-friendliness, enhancement of social responsibility, restricted use of dyes releasing carcinogens, reduced risk of falls and injuries during donning and doffing of the undergarment.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates generally to under garments. More particularly and not exclusively, the present invention relates to underwears having improved accommodation for putting on (doning) and taking off underwear, and further enabled to absorb leaks, fluids, menstrual fluids, urinary and rectum discharges, and water when wearing in place.

### BACKGROUND OF THE INVENTION AND THE PRIOR ART

Women generally menstruate once a month for a period of three to seven days. During the menstrual period, women typically use either sanitary pads or tampons to absorb menstruation fluid and to prevent staining of garments. Women typically wear underwears not designed for menstruation. Some of the available underwears are provisioned for accommodating sanitary pads or tampons. However, both the sanitary pads and the tampons do not prevent staining of the underwears. The regular underwear does not provide the required protection from staining. The stained underwear further stains an outer garment worn by women.

Furthermore, the sanitary underwears available today are made of plastic and non-breathable materials. Therefore, these underwears are uncomfortable to wear. The underwears made of non-breathable materials increase the risk of rashes and skin problems for women wearing underwears. Moreover, the underwears made of the non-breathable materials create a bad odour. Furthermore, the design of the underwear typically used by women is not suitable for preventing leaks in susceptible areas and thereby stains both the underwear and the outer garment worn by women.

Therefore, there is an unmet need for a sanitary underwear made of breathable materials. Moreover, there is an unmet need for a sanitary underwear designed to prevent leakage of the menstrual fluid from any portion of the sanitary underwear and to prevent staining of the outer garment worn by women. Further, there is no underwear which promotes safety by preventing falls and injuries, meaning the consumer can put on and take off underwear without lifting the legs, which is the why there is need of present invention as when people lose their balance and thus fall and have injuries. The side attachable pads eliminate the need to lift the leg off the floor to ‘step’ into and out of underwear. This feature benefits senior people, medical patients, maternity patients, people suffering from Intellectual and Developmental Disabilities (IDD), differently abled, and people desiring an alternative to ‘traditional’ underwear. Furthermore, there is an unmet need for an underwear allowing air permeability, maintaining dryness, and preventing odour, rashes and infections.

There is a need for underwears which are unisex as well as regular panties. The further need for panties out to a need to promote safety when putting on and taking off underwear. In the prior art, the applicant is aware of U.S. Pat. No. 5,029,345 issued to Angheluta, et al, on Jul. 9, 1991, entitled Man's Underwear with an Integral Codpiece-like Construction. Angheluta discloses a man's garment, particularly a man's underwear garment, providing a codpiece compartment secured to a front of a trunk pad of the garment.

It is found that none of the earlier inventions and patents, taken either singly or in combination, is seen to describe the instant invention as claimed. Therefore, the inventor of the present invention proposes to resolve and surmount existent technical difficulties to eliminate the aforementioned shortcomings of the prior art.

### Objects of the Invention

Few of the Objects of the Present invention are as follows:
An object of the present invention is to provide an underwear with or without an attachable pad that prevents leaks in susceptible areas and thereby further prevents stains in both the underwear and the outer garment worn by women;
Another object of the present invention is to provide an underwear with or without an attachable pad that promotes safety by preventing falls and injuries, meaning the consumer can put on and take off underwear without lifting the legs;
Yet another object of the present invention is to provide an underwear with or without an attachable pad that provides comfort to senior people, medical patients, maternity patients, people suffering from Intellectual and Developmental Disabilities (IDD), differently abled, and people desiring an alternative to traditional underwear; and
Further object of the present invention is to provide an underwear with an attachable pad that allows air permeability, and maintains dryness thereby preventing odor, rashes and infections.

Other objects, aspects, features and goals of the present invention will be better understood from the following detailed description.

### SUMMARY OF THE PRESENT INVENTION

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

Principally, in accordance with the most preferred embodiment of the present invention, a sanitary underwear with a detachably attachable organic pad is provided. The underwear as disclosed in the present invention includes side fasteners having one piece adjustable organic pad extending from the front center belly button and passing through the center of the middle crotch area up the back center to the waistband. The fastened organic pad facilitates a plurality of benefits like reduced risk of carcinogens caused by harmful pesticides and chemicals, promotion of sustainable environment, eco-friendliness, enhancement of social responsibility, restriction on the usage of dyes releasing carcinogens, reduction in the risk of falls and injuries during donning and doffing of the undergarment, to name a few. The underwear further facilitates the absorption of leaks, perspiration, feces, stains, menstrual fluids, urinary and rectum discharges. The underwear promotes fair wages and trade. The absorbent material used in the organic pad of the underwear is either an organic cotton, or a hemp, or a bamboo, or an organic terry cloth, jute and any combination thereof. The fastener for fastening the organic pad to the waist portion of the underwear is either hook and eye, or bra attachment, or frog closure, or magnetic buttons, or buttons, or ties, or cords, or ribbons, or clamps, or snaps, or strings, or zippers, or drawstrings, or an elastic button hole, or flatweaves, or flat braids or any combination thereof.

Typically, in accordance with the most preferred embodiment of the present invention, the underwear of the present invention is capable of usage by medical patients, or patients in a hospital, or pregnant women, or wheelchair-bound patients, or person with Intellectual and Developmental Disability (IDD), or injured Veterans, or people desiring a change from the appearance of traditional underwear.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following detailed description, figures, and claims.

### BRIEF DESCRIPTION OF THE ACCOMPANYING DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein. The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 exemplarily illustrates a brief construction of an underwear for preventing staining of an outer garment of a wearer by a body fluid of the wearer.

FIG. 2 exemplarily illustrates the back structure of the fabric body.

FIG. 3 exemplarily illustrates the properties of the fabric from the front;

FIG. 4 exemplarily illustrates a back view of the brief construction of the underwear;

FIG. 5A through FIG. 5B exemplarily illustrates an open view of the attachable pad construction of the underwear with stitches in the rear portion; and

FIG. 6 exemplarily illustrates the open view of the brief construction of the underwear in the open portion.

### DETAILED DESCRIPTION OF THE PREFERRED EMBODIMENTS OF THE PRESENT INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

There are four major parts; 1) front, 2) middle or crotch, and 3) back, and 4) attached pad that goes from the front center belly button pass across the crotch/middle section up the back rear center waistband. One pad (either front or rear, respectively) is secured about the pelvis area, the panties are carried across the crotch area to the other pad (either rear or front, respectively) and secured in place by using different means, methods, mechanism, fabric, accessories, and fasteners, etc.

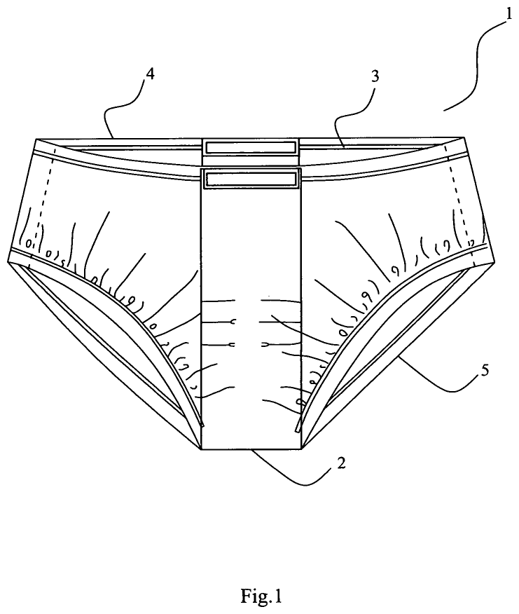

Referring now to FIG. 1, there is an illustration of the front appearance of the underwear 1 in accordance with the most preferred embodiment. The underwear 1 comprises a detachably attachable organic pad 2, the front side of which is illustrated in the drawings with side closure showing the front appearance of underwear 1 with “U” shape. The detachably attachable organic pad 2 is adapted to cover the crotch. The detachably attachable organic pad 2 can be removed, washed, and reused or discarded. The purpose of adapting the detachably attachable organic pad 2 is to absorb leaks, fluids, discharges, urine, faeces, menstrual fluids, stains, and the like. There are pleats and elastic 3 around the waist 4 and leg opening 5. The detachably attachable organic pad 2 has pleats (not shown in the drawings) on the front sides to expand for unisex underwear. There is a place on the side for various means, methods, mechanism, devices, fabrics, and accessories such as hook and eye, bra attachments, frog closure, magnetic buttons, buttons, ties, cords, ribbons, clamps, snaps, strings, zippers, drawstrings, and the like.

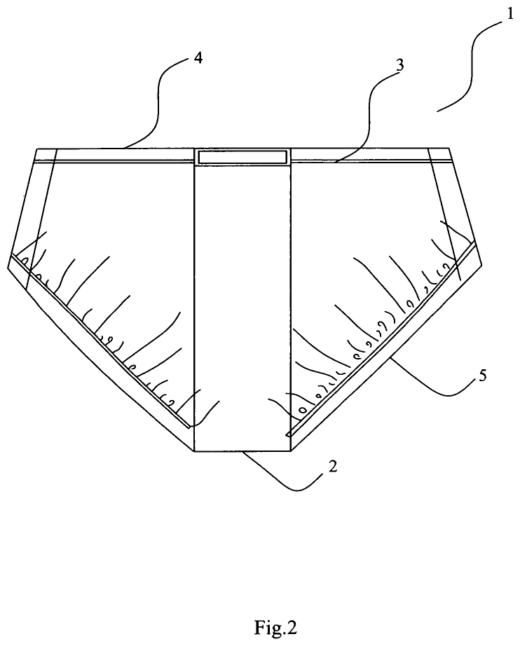

Referring now to FIG. 2, there is an illustration of the back appearance of the underwear 1 in accordance with the most preferred embodiment. The underwear 1 comprises an a detachably attachable organic pad 2, the front side side of which is illustrated in the drawings with side closure showing the back appearance of underwear 1 with “U” shape. The detachably attachable organic pad 2 can be removed, washed, and reused or discarded. The purpose of adapting the detachably attachable organic pad 2 is to absorb leaks, fluids, discharges, urine, faeces, menstrual fluids, stains, and the like. There are pleats and elastic 3 around the waist 4 and leg opening 5. The detachably attachable organic pad 2 has pleats (not shown in the darwings) on the front sides to expand for unisex underwear. There is a place on the side for various means, methods, mechanism, devices, fabrics, and accessories such as hook and eye, bra attachments, frog closure, magnetic buttons, buttons, ties, cords, ribbons, clamps, snaps, strings, zippers, drawstrings, and the like.

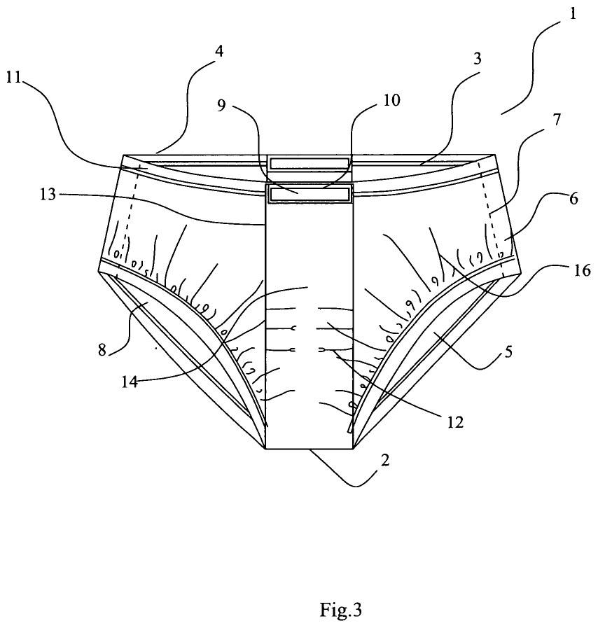

Referring now to FIG. 3, there is an illustration of the properties of the detachably attachable organic pad 2 in the front view of the underwear 1. In accordance with the most preferred embodiment of the present invention, the underwear comprises a dyed to match (DTM) Velcro 6 along the side seams 7 of the underwear 1. The edges of the leg covering portion 8 on either side of the underwear 1 comprises ¼″ hem made from double needle cover stitch. As an example, a Velcro 9 is shown for fixing the detachably attachable organic pad 2 with the front center 10 of the waist portion 11 of the underwear 1. The detachably attachable organic pad 2 is preferably having a width of 3⅞″. There is a set of horizontal tucks 12 extended from the vertical side 13 of the detachably attachable organic pad 2 provided on the outer surface 14. Evenly distributed gathering 16 is also shown in the underwear 1 on either side of the detachably attachable organic. pad 2. The detachably attachable organic pad 2 can be removed, washed, and reused or discarded. The purpose of adapting the detachably attachable organic pad 2 is to absorb leaks, fluids, discharges, urine, feces, menstrual fluids, stains, and the like. There are pleats and elastic 3 around the waist 4 and leg opening 5. The detachably attachable organic pad 2 has pleats on the front sides to expand for unisex underwear. There is a place on the side for various means, methods, mechanism, devices, fabrics, and accessories such as hook and eye, bra attachments, frog closure, magnetic buttons, buttons, ties, cords, ribbons, clamps, snaps, strings, zippers, drawstrings, and the like.

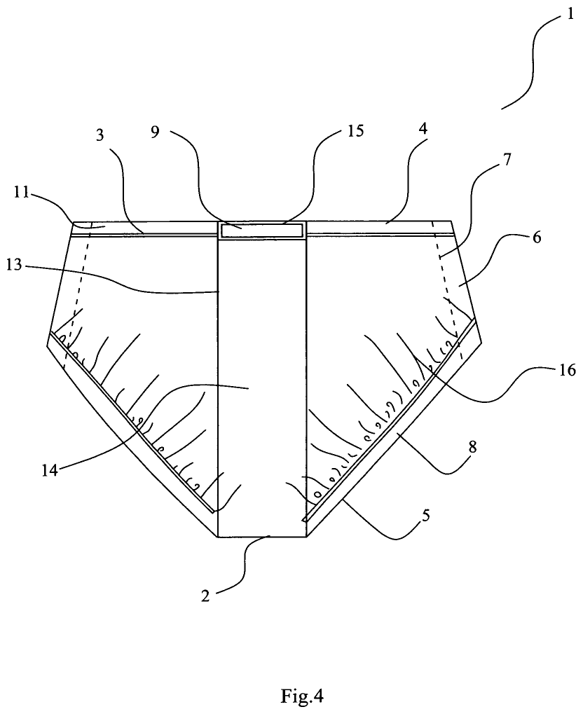

Referring now to FIG. 4, there is an illustration of back view of the brief construction of the underwear 1 in accordance with the most preferred embodiment of the present invention. In accordance with the most preferred embodiment of the present invention, the underwear comprises a dyed to match (DTM) Velcro 6 along the side seams 7 of the underwear 1. The edges of the leg covering portion 8 on either side of the underwear 1 comprises ¼″ hem made from double needle cover stitch. As an example, a Velcro 9 is shown for fixing the detachably attachable organic pad 2 with the back center 15 of the waist portion 11 of the underwear 1. The detachably attachable organic pad 2 is preferably having a width of 3⅞″. There is a set of horizontal tucks (not shown in the drawings) that extended from the vertical side 13 of the detachably attachable organic pad 2 provided on the outer surface 14 of the pad. Evenly distributed gathering 16 is also shown in the underwear 1 on either side of the detachably attachable organic pad 2. The detachably attachable organic pad 2 can be removed, washed, and reused or discarded. The purpose of adapting the detachably attachable organic pad 2 is to absorb leaks, fluids, discharges, urine, faeces, menstrual fluids, stains, and the like. There are pleats and elastic around the waist 4 and leg opening 5. The detachably attachable organic pad 2 has pleats on the front sides to expand for unisex underwear. There is a place on the side for various means, methods, mechanism, devices, fabrics, and accessories such as hook and eye, bra attachments, frog closure, magnetic buttons, buttons, ties, cords, ribbons, clamps, snaps, strings, zippers, drawstrings, and the like.

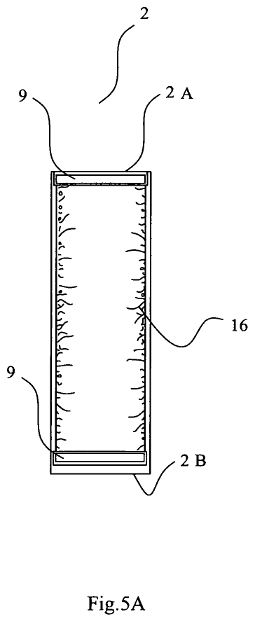

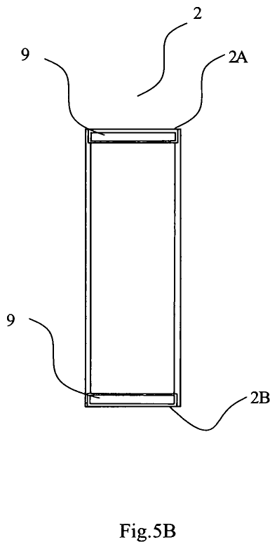

Referring now to FIG. 5A, there is an illustration of an open view of the construction of the detachably attachable organic pad 2 of the underwear 1 having gathering 16 and further having stitches in the rear portion is illustrated. The detachably attachable organic pad 2, at the endpoints 2A and 2B along its length, is having a ½″ dyed to match [DTM] fastener 9 positioned inside the organic pad, which herein is taken as Velcro fastener as an example. The Velcro fastener 9 is adapted to detachably attach the organic pad 2 to the front center 10 and back center 15 (as shown in FIG. 3 and FIG. 4 respectively) of the waist portion 11 of the underwear 1. The horizontal length and width of the dyed to match [DTM] Velcro fastener 9 are respectively 3⅞″ and 1.2″. The total end-to-end length of the detachably attachable organic pad 2 is preferably 23″. The detachably attachable organic pad 2 comprises layers of flat absorbent material preferably three layers of flat absorbent material with an evenly distributed gathering 16 thereon. These detachably attachable organic pads 2 appearing in the “U” shape can be washed, dried, reused or discarded. The detachably attachable organic pad 2 may be made double, triple, or quadruple in size to increase absorbency.

Referring now to FIG. 5B, there is an illustration of an open view of the construction of the detachably attachable organic pad 2 of the underwear 1 without any evenly distributed gathering and further having stitches in the rear portion is illustrated. The detachably attachable organic pad at the end points 2A and 2B along its length, is having a ½″ dyed to match [DTM] fastener 9 positioned inside the organic pad, which herein is taken as Velcro fastener as an example. The Velcro fastener 9 is adapted to detachably attach the organic pad 2 to the front center 10 and back center 15 (as shown in FIG. 3 and FIG. 4 respectively) of the waist portion 11 of the underwear 1. The horizontal length and width of the dyed to match [DTM] Velcro fastener 9 are respectively 3⅞″ and 1.2″. The total end-to-end length of the detachably attachable organic pad 2 is preferably 23″. The detachably attachable organic pad 2 comprises layers of flat absorbent material preferably three layers of flat absorbent material without any evenly distrinuted gathering thereon. The detachably attachable organic pad 2 appearing in the “U” shape can be washed, dried, reused or discarded. The detachably attachable organic pad 2 may be made double, triple, or quadruple in size to increase absorbency.

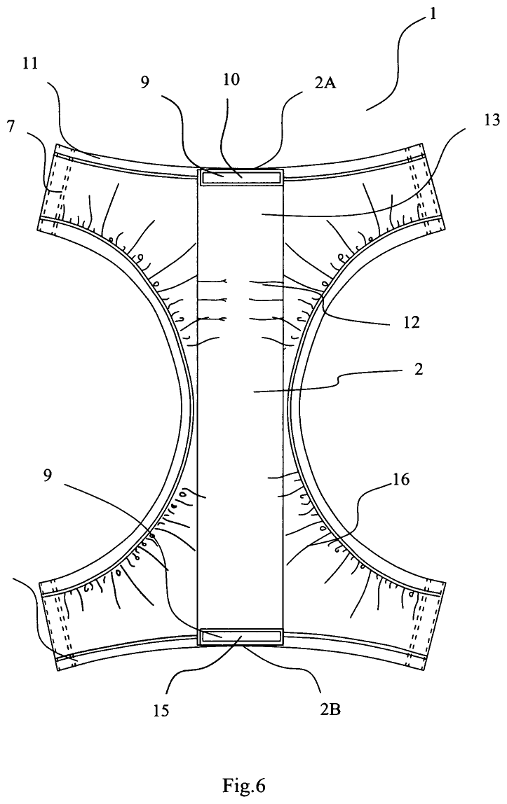

Referring now to FIG. 6 there is an illustration of the open view of the brief construction of the underwear 1 in the open portion. The detachably attachable organic pad 2 has a length of 23″ and a width of 3⅝″. The detachably attachable organic pad 2, at the end points 2A and 2B along its length, is having a ½″ dyed to match [DTM] fastener 9 positioned inside the organic pad. The fastener 9 is taken as Velcro fastener as an example. The Velcro fastener 9 is adapted to detachably attach the organic pad 2 to the front center 10 and back center 15 of the waist portion 11 of the underwear 1. The horizontal length and width of the dyed to match [DTM] Velcro fastener 9 are respectively 3⅞″ and 1.2″ respectively. The total end-to-end length of the detachably attachable organic pad 2 is preferably 23″. The detachably attachable organic pad 2 comprises preferably there layers of flat absorbent material with an evenly distributed gathering 16 thereon. There is provided a set of three horizontal tucks 12 on the detachably attachable organic pad 2 on its outer surface with the first tuck positioned at a distance of 4.5″ below the waist band in the waist portion 11. Any two consecutive tucks in the set of tucks 12 are separated by a distance of ⅝″. The detachably attachable organic pad 2 appearing in the “U” shape can be washed, dried, reused or discarded. The detachably attachable organic pad 2 may be made double, triple, or quadruple in size to increase absorbency.

The detachably attachable organic pad 2 is made of cotton, hemp, bamboo or jute. The detachably attachable organic pad 2 is fixed in the center of the underwear 1. The detachably attachable organic pad 2 is designed to absorb fluids, liquids, leaks, discharges, urine, faeces, menstrual fluids, stains, and more.

The open flat underwear has pleats for unisex reasons. To put detachably attachable organic pad 2 on this underwear, completely open the underwear on a flat surface, such as the corner of a bed or stable chair. Sit your rear human bottom/butt directly on the back pad, reach between the legs to pull the front pad across the urinary/pelvis area. The middle pad 2 should cover the human crotch area. Close the underwear, by utilizing the side closures to securely complete the dressing process before standing up from the bed or chair.

The underwear 1 as construed by the present invention and illustrated in the drawings of FIG. 1 through FIG. 6 will promote safety by reducing falls and injuries when donning and doffing traditional panties that require an individual to lift each foot at a different time to put on and take off panties. When a person is wearing multiple layers of clothes, such as a girdle, panties hose, or/and hosiery, these panties can be changed/removed/replaced without ‘stepping out’ or ‘stepping into’ them when wearing multiple layers.

The underwear 1 as construed by the present invention and illustrated in the drawings of FIG. 1 through FIG. 6 with frog eyelet fasteners with the one piece adjustable organic cotton pad 2 that extends from the front center belly button that passes across the center middle crotch area, up the back center to the waistband is a unique and novel concept made from organic cotton, hemp, bamboo, and/or organic terry cloth can absorb leaks, perspiration, menstrual fluids, and rectum discharges.

The sanitary underwear in accordance with the most preferred embodiment of the present invention drastically promotes safety by reducing lower torso movement when lifting a foot off the floor to step into a traditional panty leg opening.

The center pad that is made from organic cotton, hemp, bamboo, and/or other organic terry cloth fabrics that can absorb leaks, fluids, menstrual fluids, urinary, and rectum discharges, and water when wearing in place. The versatile center pad can be removed, washed, discarded, or replaced as needed.

Organic cotton that has been certified will reduce carcinogens and reduce the risk of that can be absorbed in the skin. Organic cotton reduces harmful chemicals and pesticides that can lead to long term medical complications and hospitalization.

The sanitary underwear in accordance with the most preferred embodiment of the present invention drastically promotes safety while reducing falls and injuries when donning and doffing underwear in a sitting position.

The sanitary underwear in accordance with the most preferred embodiment of the present invention reduces falls and injuries for seniors are vital. The most common injury to seniors occurs because of falls and injuries, such as a broken bone from a fall.

Injured Veterans with medical challenges will benefit from the ease of putting on and taking off this underwear.

Medical patients will benefit from this underwear because this underwear requires minimum lower torso movement to put on and take off. Hospital patients can put on and take off this underwear by ‘opening’ this underwear on a hospital bed, so the underwear is flat in the center of the bed.

The hospital patient can turn on the side to start the process of putting on this underwear, then turn to the opposite side to complete the robe process.

Maternity patients, who cannot see where to step into traditional underwear will prevent falls and possible injuries to the fetus and mom-to-be. Some people might want a change from traditional underwear that requires lifting the legs and maintaining the balance when getting dress. Some people will want a change from the appearance of traditional underwear.

While a specific embodiment has been shown and described, many variations are possible. With time, additional features may be employed. The particular shape or configuration of the platform or the interior configuration may be changed to suit the system or equipment with which it is used.

Wheelchair patients will benefit from the use of this underwear due to reduced lower torso movement. This underwear can be placed completely open with the rear pad in the wheelchair. The person can seat in the wheelchair on the rear underwear pad and pull the front pad in between the legs and attached on each side.

Intellectual and Developmental Disability (IDD) society will benefit from the use of this underwear due to reduce lower torso movement and ease of muscle movement with the hook and eye, magnetic button, and more closures. This underwear can be placed completely open on the middle-edge of a bed, center of a stable chair, or a secure, stable, and safe location. Once the underwear is completely open, the IDD person will sit on the rear underwear pad and pull the front pad in between the legs across the crotch/pad area and close the underwear on each side.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The abstract of the disclosure is provided to allow the reader to quickly ascertain the nature of the technical disclosure. It is submitted with the understanding that it will not be used to interpret or limit the scope or meaning of the claims. In addition, in the foregoing detailed description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the detailed description, with each claim standing on its own as a separately claimed subject matter.

These underwears are unisex as well as regular panties. I have invented these panties out to a need to promote safety when putting on and taking off underwear.

Technical Advantages and Economic Significance of the Present Invention

The present invention provides a sanitary underwear with adjustable organic pad. The underwear in accordance with the preferred embodiment of the present invention prevents leaks in susceptible areas and thereby further prevents stains in both the underwear and the outer garment worn by women. The underwear promotes safety by preventing falls and injuries, meaning the user can put on and take off underwear without lifting the legs. The underwear of the present invention with adjustable organic pad provides comfort to senior people, medical patients, maternity patients, people suffering from Intellectual and Developmental Disabilities (IDD), differently abled, and people desiring an alternative to ‘traditional’ underwear. Further, the adjustable organic pad of the underwear of the present invention allows air permeability and maintains dryness thereby preventing odour, rashes and infections.

Many modifications and other embodiments of the invention will come to the mind of one skilled in the pertinent art having the benefit of teaching presented in the foregoing descriptions and the associated drawings. Therefore, it is understood that the invention is not limited to the specific embodiments disclosed and that modifications and variations of the disclosed embodiments are intended to be included as readily appreciated by those skilled in the pertinent art. It is contemplated that these modifications and variations be included within the scope and ambit of the instant invention which is defined by the following claims.

1) I claim that the attachable organic cotton panties comprising side fasteners without the one piece adjustable organic cotton panel that extends from the front centre belly button pass the centre middle crotch area up the back centre to the waistband is a unique and novel concept that 1) decrease the risk of carcinogens that are caused by harmful pesticides and chemicals, 2) promotes a sustainable environment, 3) is eco-friendly to the environment, 4) social responsibilities are enhanced, 5) dyes that release carcinogens are restricted, 6) the risk of falls and injuries are reduced when donning and doffing underwear due to less lower torso movements 7) the risk of falls and injuries are reduced when donning and doffing underwear in a sitting position

2) further comprise, according to claim 1, wherein said attachable organic cotton panties with the hook and eye fasteners with the one piece adjustable organic cotton panel that extends from the front centre belly button that passes across the centre middle crotch area, up the back centre to the waistband is a unique and novel concept made from organic cotton, hemp, bamboo, and/or organic terry cloth can absorb leaks, perspiration, menstrual fluids, and rectum discharges.

3) further comprise, according to claim 1, wherein said attachable organic cotton panties with the bra extenders hook and attachments fasteners without the one piece adjustable organic cotton panel that extends from the front centre belly button pass the centre middle crotch area up the back centre to the waistband

4) further comprise, according to claim 1, wherein said attachable organic cotton panties with the elastic button holes fasteners with the one piece adjustable organic cotton panel that extends from the front centre belly button that passes across the centre middle crotch area, up the back centre to the waistband is a unique and novel concept made from organic cotton, hemp, bamboo, and/or organic terry cloth can absorb leaks, perspiration, menstrual fluids, and rectum discharges.

5) further comprise, according to claim 1, wherein said attachable organic cotton panties with the drawstrings fasteners without the one piece adjustable organic cotton panel that extends from the front centre belly button pass the centre middle crotch area up the back centre to the waistband

6) further comprise, according to claim 1, wherein said attachable organic cotton panties with the zipper fasteners with the one piece adjustable organic cotton panel that extends from the front centre belly button that passes across the centre middle crotch area, up the back centre to the waistband is a unique and novel concept made from organic cotton, hemp, bamboo, and/or organic terry cloth can absorb leaks, perspiration, menstrual fluids, and rectum discharges.

7) further comprise, according to claim 1, wherein said attachable organic cotton panties with the button fasteners without the one piece adjustable organic cotton panel that extends from the front centre belly button pass the centre middle crotch area up the back centre to the waist.

8) further comprise, according to claim 1, wherein said attachable organic cotton panties with frog eyelet closure fasteners without the one piece adjustable organic cotton panel that extends from the front centre belly button pass the centre middle crotch area up the back centre to the waistband

9) further comprise, according to claim 1, wherein said attachable organic cotton panties with lace tie-up fasteners without the one piece adjustable organic cotton panel that extends from the front centre belly button pass the centre middle crotch area up the back centre to the waistband.

10) further comprise, according to claim 1, wherein said attachable organic cotton panties with the snap closure/fasteners without the one piece adjustable organic cotton panel that extends from the front centre belly button pass the centre middle crotch area up the back centre to the waistband is a unique and novel concept

11) further comprise, according to claim 1, wherein said attachable organic cotton panties with cord fasteners without the one piece adjustable organic cotton panel that extends from the front centre belly button pass the centre middle crotch area up the back centre to the waistband is a unique and novel concept.

12) further comprise, according to claim 1, wherein said attachable organic cotton panties with strings and threads fasteners without the one piece adjustable organic cotton panel that extends from the front centre belly button pass the centre middle crotch area up the back centre to the waistband is a unique and novel concept.

13) I claim that the attachable organic cotton panties with magnetic buttons fasteners without the one piece adjustable organic cotton panel that extends from the front centre belly button pass the centre middle crotch area up the back centre to the waistband is a unique and novel concept.

14) further comprise, according to claim 2, wherein said attachable organic cotton panties with various methods, means, mechanics, mechanism, and ways through fasteners without the one piece adjustable organic cotton panel that extends from the front centre belly button pass the centre middle crotch area up the back centre to the waistband is a unique and novel concept.

15) further comprise, according to claim 2, wherein said attachable organic cotton panties with button holes and ties/cords, flat braid, and flatweave fasteners without the one piece adjustable organic cotton panel that extends from the front centre belly button pass the centre middle crotch area up the back centre to the waistband is a unique and novel concept.

16) further comprise, according to claim 2, wherein said attachable crotch less organic cotton panties with fasteners is a unique and novel concept for medical, urinary, rectum, menstruating, outpatient, and hospital patients will benefit from these panties due to medical reason and these panties: These panties can be used in an intimate setting.

17) I claim that the benefit to wearing certified organic cotton underwear is to reduce health problems caused by harmful chemicals and pesticides. Organic cotton underwear will promote a safer ego-friendly environment with fair wages and trade.
